(19) United States
(12) Patent Application Publication
(10) Pub. No.: US 2020/0237235 A1
(43) Pub. Date: Jul. 30, 2020

(54) ANALYZER AND METHOD FOR ANALYZING MICROVASCULAR HEMODYNAMIC PARAMETERS

(71) Applicant: Xuzhou Cardiovascular Institute, Xuzhou (CN)

(72) Inventors: Haibin Gong, Xuzhou (CN); Xingxin Chen, Xuzhou (CN); Bingquan Luo, Xuzhou (CN)

(21) Appl. No.: 16/387,706

(22) Filed: Apr. 18, 2019

(30) Foreign Application Priority Data
Jan. 28, 2019 (CN) 201910080819.6

(51) Int. Cl. A61B 5/02; A61B 5/021; A61B 5/0235; A61B 5/026; G02B 21/36

(52) U.S. Cl. CPC A61B 5/02007; A61B 5/02141; G02B 21/365; A61B 5/0261; A61B 5/0235

(57) ABSTRACT

An analyzer and analysis method are provided for analyzing microvascular hemodynamic parameters. The analyzer includes a capillary-pressure detection device, a segmental blood pressure measuring device, a microcirculation microscopic image capturing device, and a data processing and analyzing module. The capillary-pressure detection device is connected to the data processing and analyzing module to measure a capillary pressure and obtain a capillary-pressure signal. The segmental blood pressure measuring device is connected to the data processing and analyzing module to obtain a segmental-blood signal. The microcirculation microscopic image capturing device is connected to the data processing and analyzing module to acquire a static circulation image and a dynamic circulation image. The data processing and analyzing module is used to obtain hemodynamic parameters from each of these signals and images and then plot a Safari blood pressure curve, thereby achieving the acquisition and analysis of the hemodynamic parameters.

### CROSS-REFERENCE TO RELATED APPLICATION

This application claims priority to Chinese application number 201910080819.6, filed on Jan. 28, 2019. The above-mentioned patent application is incorporated herein by reference in its entirety.

### TECHNICAL FIELD

The present invention relates to the field of medicine, and more particularly, to a device and method for analyzing microvascular hemodynamic parameters.

### BACKGROUND

Hypertension is a chronic disease that is the leading cause of stroke, myocardial infarction, heart failure, cardiovascular diseases, chronic kidney diseases, and premature death. The number of hypertensive patients in China is 290 million, with the prevalence rate being 25%, and the control rate being less than 10%. Hypertension is a cardiovascular syndrome caused by the interaction between genes and environmental factors. Primary hypertension refers to the hypertension of which no identifiable cause is found, that accounts for about 90% of the hypertension group, but for which the pathogenesis of elevated blood pressure is clear. Arterial hypertension refers to persistently elevated blood pressure (BP), which is the product of cardiac output (CO) and peripheral vascular resistance (PVR) (BP=CO×PVR).

Hypertension is ultimately a hemodynamic abnormality, and the hemodynamic characteristics of hypertension are elevated blood pressure, increased arterial stiffness and increased peripheral vascular resistance. Description of the intercardiovascular mechanical pressure should focus on two components, i.e., pulse pressure (PP) of pulse oscillation and stable and continuous mean arterial pressure (MAP).

PP plays the dominant role as an elastic property of the aorta. The composition of the aortic includes an endothelium layer, vascular smooth muscle cells, and a large number of extracellular elastic tissues. This structure provides a transient buffering function (windkkessel) for the cardiac ejection phase. A hypertensive patient has increased arterial stiffness and wave reflection, increased central SBP, PP and wide PP, excessive outward arterial stress (homonymous stress, shear stress) and arterial conduction velocity (gradient reversal), which damages the endothelium cells of a resistance vascular in a target organ and myogenic tension (increasing the glomerular filtration rate and proteinuria).

MAP is determined by the blood resistance of smaller arteries and arterioles. These distal small arteries and arterioles are composed of continuous endothelium layers, a large number of smooth muscle cell layers, and fewer extracellular elastic tissues. A large pressure drop occurs in this resistance vascular, and the arterial effect also gradually disappears. This high transmural pressure in the hypertensive patient is regulated by a myogenic response, which produces a pressure wave reflection to ensure that a steady pressure flow state is achieved in the microcirculation. However, the chronic myogenic tension is reconstituted inward nutritionally, with reduced perfusion and sparse micro vessels. Sparse blood capillaries and structurally increased peripheral resistance, are also the initiating mechanism of primary hypertension (at least for parts of the patients with primary hypertension). The relationship between hemodynamics related to pressure, flow and resistance is as follows: 1. a blood flow (F) is directly proportional to a pressure (P) and a radius of a blood vessel (r), and is inversely proportional to resistance (R); 2. the resistance (R) is directly proportional to the blood pressure viscosity (η) and the length of the blood vessel (l), and is inversely proportional to the fourth power of the vascular radius (r), and the resistance of a single blood vessel R=MAP/F; 3. most of the microcirculation is a network structure, and the structural system of the network resistance vascular is a determining factor, the number of blood vessels and their connections, such as parallel coupling of blood vessels, or arched tandem form of blood vessels, etc.; and 4. a peripheral segmental vascular pressure drop represents the blood resistance in this segment of blood vessel. It can be seen that, the microvascular hemodynamic parameters can reflect the mechanism of hypertension, but it is an urgent technical problem to be solved how to obtain the microvascular hemodynamic parameters.

Therefore, it would be desirable to provide an analyzer for analyzing microvascular hemodynamic parameters and an analysis method for the same, so as to obtain microvascular hemodynamic parameters.

### SUMMARY

To achieve the above purposes and solve the technical defects with the conventional methods, the present invention provides the following technical solution, in one embodiment. An analyzer for analyzing microvascular hemodynamic parameters includes a capillary-pressure detection device, a segmental blood pressure measuring device, a microcirculation microscopic image capturing device, and a data processing and analyzing module. The capillary-pressure detection device is connected to the data processing and analyzing module to measure a capillary pressure, obtain a capillary-pressure signal, and output the capillary-pressure signal to the data processing and analyzing module. The segmental blood pressure measuring device is connected to the data processing and analyzing module to acquire a finger-artery blood pressure, a wrist-artery blood pressure and a brachial-artery blood pressure, obtain a segmental-blood signal, and output the segmental-blood signal to the data processing and analyzing module. The microcirculation microscopic image capturing device is connected to the data processing and analyzing module to acquire a static circulation image and a dynamic circulation image, and output the static circulation image and the dynamic circulation image to the data processing and analyzing module. Finally, the data processing and analyzing module is used to acquire hemodynamic parameters from the capillary-pressure signal, the segmental-blood signal, the static circulation image and the dynamic circulation image, and plot a Safari blood pressure curve. The hemodynamic parameters include a capillary pressure Pₘ, a capillary blood flow velocity v, a capillary blood flow volume, a capillary local blood flow volume, a capillary resistance R, a segmental vascular pressure, and a segmental vascular pressure difference.

In one aspect, the capillary-pressure detection device includes a precision precession device, a precision lever, a capillary-pressure measuring device, a precision pressure sensor, and a signal acquisition and amplification circuit. The precision precession device is connected to one end of the precision lever, the precision pressure sensor is disposed at one end of the precision lever, the capillary-pressure measuring device is disposed at the other end of the precision lever, and the precision precession device is used for pulling up one end of the precision lever, such that the other end of the precision lever squeezes the capillary on the capillary-pressure measuring device. An output end of the precision pressure sensor is connected to an input end of the signal acquisition and amplification circuit, and the precision pressure sensor measures the capillary pressure of the capillary according to a mechanical lever principle, obtains a capillary-pressure analog signal, and output the capillary pressure analog signal to the signal acquisition and amplification circuit. An output end of the signal acquisition and amplification circuit is connected to the data processing and analyzing module, and the signal acquisition and amplification circuit is used to conduct amplifying and digital processing of the capillary pressure analog signal, obtain a capillary-pressure signal, and output the capillary-pressure signal to the data processing and analyzing module.

In another aspect, the precision precession device includes a precision screw and a precision nut.

In a further aspect, the microcirculation microscopic image capturing device includes a microscope and a camera, one end of the microscope directly faces the capillary-pressure measuring device of the capillary-pressure detection device, the camera is connected to the data processing and analyzing module, the other end of the microscope directly faces the camera. The microscope is used for amplifying the capillary to obtain an enlarged capillary, the camera is used for acquiring a static circulation image and a dynamic circulation image of the enlarged capillary, and output the static circulation image and the dynamic circulation image to the data processing and analyzing module.

In yet another aspect, the segmental blood pressure measuring device includes a controller, an electronic valve, and multiple cuffs. The controller is connected to a control end of the electronic valve, an output end of the electronic valve is connected to the multiple cuffs respectively, and the controller is used for controlling the on-off actuation state of the electronic valve, and in turn controlling the multiple cuffs to conduct sequential measurement of a finger-artery blood pressure, a wrist-artery blood pressure and a brachial-artery blood pressure. The multiple cuffs are respectively connected to the controller, the multiple cuffs are used for measuring the finger-artery blood pressure, the wrist-artery blood pressure and the brachial-artery blood pressure, and outputting the finger-artery blood pressure, the wrist-artery blood pressure and the brachial-artery blood pressure to the controller. The controller is connected to the data processing and analyzing module, and the controller is used for outputting the finger-artery blood pressure, the wrist-artery blood pressure and the brachial-artery blood pressure to the data processing and analyzing module.

In accordance with another embodiment of the invention, a method for analyzing microvascular hemodynamic parameters includes the following steps: acquiring a capillary-pressure signal, and acquiring a capillary pressure Pₘ based on the capillary-pressure signal; acquiring a segmental-blood signal, and acquiring a segmental vascular pressure difference based on the segmental-blood signal, and acquiring information about blood resistances R in respective segments based on the segmental vascular pressure difference; acquiring a dynamic circulation image, and calculating a capillary blood flow velocity v based on the dynamic circulation image by using an automatic stepping method; and plotting a Safari blood pressure curve according to the capillary pressure Pₘ, respective segmental vascular resistances R and the capillary blood flow velocity v.

In one aspect, the step of calculating the capillary blood flow velocity v based on the dynamic circulation image by using an automatic stepping method specifically includes: acquiring a marker appeared in the dynamic circulation image; determining the original coordinates of the marker; delineating the motion track of the marker; playing back the dynamic circulation image frame by frame, and tracking the coordinate of the marker when the image is played back to the Nth frame based on the motion track, so as to obtain the coordinate of the marker in the Nth frame; calculating a distance L between the original coordinate and the coordinate of the marker in the Nth frame; and calculating a capillary blood flow velocity v utilizing an equation v=L/(N·t₀) based on the distance, where t₀ is the image acquisition time per frame.

In another aspect, after acquiring the dynamic circulation image, and calculating the capillary blood flow velocity v based on the dynamic circulation image by using an automatic stepping method, the method further includes: acquiring a static circulation image, and measuring a vascular diameter D and a vascular length based on the static circulation image, and delineating a vascular network.

In a further aspect, after acquiring the static circulation image, and measuring the vascular diameter D and the vascular length based on the static circulation image, and delineating the vascular network, the method further includes: obtaining a vascular density Dₙ, a dynamic to static ratio K, and a replacement index P based on the vascular network.

In yet another aspect, after obtaining the vascular density Dₙ, the dynamic to static ratio K, and the replacement index P based on the vascular network, the method further includes: calculating a single-blood-vessel blood flow volume Q utilizing an equation Q=v(πD²/4) based on the blood flow velocity v and the vascular diameter D; and calculating a local blood flow volume Qᵤ utilizing an equation Qᵤ=Q·Dₙ based on the single-blood-vessel blood flow volume Q and the vascular density Dₙ.

The embodiments of the present invention provide the following technical effects. The present invention provides an analyzer for analyzing microvascular hemodynamic parameters and an accompanying analysis method. The analyzer includes: capillary-pressure detection device, a segmental blood pressure measuring device, a microcirculation microscopic image capturing device, and a data processing and analyzing module; where the capillary-pressure detection device is connected to the data processing and analyzing module to measure a capillary pressure and obtain a capillary-pressure signal; the segmental blood pressure measuring device is connected to the data processing and analyzing module to obtain a segmental-blood signal; the microcirculation microscopic image capturing device is connected to the data processing and analyzing module to acquire a static circulation image and a dynamic circulation image; and the data processing and analyzing module is used to obtain hemodynamic parameters from the capillary-pressure signal, the segmental-blood signal, the static circulation image and the dynamic circulation image, thereby achieving the acquisition of the hemodynamic parameters.

In embodiments of the present invention, the microcirculation function state is studied from three aspects, i.e., the capillary pressure, the capillary blood flow velocity and the capillary resistance, which changes the drawbacks of the previous analysis of microcirculation function based on a single index. Due to the lack of a capillary-pressure index in the past, the microcirculation function is evaluated only by indexes such as the vascular network and the blood flow velocity during the analysis of microcirculation. Therefore, the analysis of a comprehensive hemodynamic index is lacked, and there is a limitation in the observation index. For example, in terms of indexes of the blood flow velocity, high blood flow and low blood pressure (low resistance) is conducive to blood perfusion, and is beneficial to microcirculation, while high blood flow and high blood pressure (high resistance) increases the blood perfusion in form, but increases the damage to the blood vessel, and thus is harmful to the microcirculation.

### BRIEF DESCRIPTION OF THE DRAWINGS

Various additional features and advantages of the invention will become more apparent to those of ordinary skill in the art upon review of the following detailed description of one or more illustrative embodiments taken in conjunction with the accompanying drawings. The accompanying drawings, which are incorporated in and constitutes a part of this specification, illustrate one or more embodiments of the invention and, together with the general description given above and the detailed description given below, explain the one or more embodiments of the invention.

FIG. 1 is a schematic structural view of an analyzer for analyzing microvascular hemodynamic parameters in accordance with one embodiment of the present invention.

FIG. 2 is a schematic structural view of a capillary-pressure detection device and a microcirculation microscopic image capturing device of an analyzer according to another embodiment.

FIG. 3 is a graphical plot in the form of a Safari curve diagram of portions of the analyzer of FIG. 2.

FIG. 4 is a flow chart showing steps of a method for analyzing microvascular hemodynamic parameters in accordance with another embodiment consistent with the embodiment of FIGS. 2 and 3.

### DETAILED DESCRIPTION

The following clearly and completely describes the technical solutions in the embodiments of the present invention with reference to the accompanying drawings in the embodiments of the present invention. To make objectives, features, and advantages of the present invention clearer, the following describes embodiments of the present invention in more detail with reference to accompanying drawings and specific implementations.

### Embodiment 1

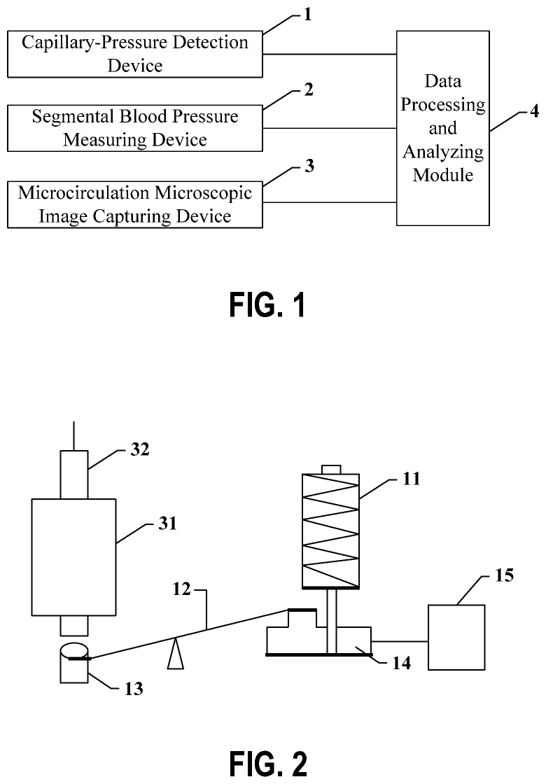

An analyzer is provided in one embodiment for analyzing microvascular hemodynamic parameters. As shown in FIG. 1, the analyzer includes: a capillary-pressure detection device 1, a segmental blood pressure measuring device 2, a microcirculation microscopic image capturing device 3, and a data processing and analyzing module 4. The capillary-pressure detection device 1 is connected to the data processing and analyzing module 4 to measure a capillary pressure and obtain a capillary-pressure signal, and output the capillary-pressure signal to the data processing and analyzing module 4. The segmental blood pressure measuring device 2 is connected to the data processing and analyzing module 4 to acquire a finger-artery blood pressure, a wrist-artery blood pressure and a brachial-artery blood pressure, obtain a segmental-blood signal, and output the segmental-blood signal to the data processing and analyzing module 4. The microcirculation microscopic image capturing device 3 is connected to the data processing and analyzing module 4 to acquire a static circulation image and a dynamic circulation image, and output the static circulation image and the dynamic circulation image to the data processing and analyzing module 4. The data processing and analyzing module 4 is used to acquire hemodynamic parameters from the capillary-pressure signal, the segmental-blood signal, the static circulation image and the dynamic circulation image, and then plot a Safari blood pressure curve. The hemodynamic parameters include a capillary pressure Pₘ, a capillary blood flow velocity v, a capillary blood flow volume, a capillary local blood flow volume, a capillary resistance R, a segmental vascular pressure, and a segmental vascular pressure difference.

### Embodiment 2

In another embodiment, an analyzer is provided for analyzing microvascular hemodynamic parameters. As shown in FIG. 2, the capillary-pressure detection device 1 includes a precision precession device 11, a precision lever 12, a capillary-pressure measuring device 13, a precision pressure sensor 14, and a signal acquisition and amplification circuit 15. The capillary-pressure measuring device 13 is disposed at one end of the precision lever 12, the precision pressure sensor 14 is disposed at the other end of the precision lever 12, and the other end of the precision lever 12 is also connected to the precision precession device 11. An output end of the precision pressure sensor 14 is connected to an input end of the signal acquisition and amplification circuit 15. The output end of the signal acquisition and amplification circuit 15 is connected to the data processing and analyzing module 4. During use, the precision sensor at the other end of the precision lever 12 is gradually pulled up through the precision precession device 11, the one end of the precision lever 12 squeezes the capillary, the precision precession device 11 is adjusted to transfer the capillary pressure through the precision lever 12 to the precision pressure sensor 14 (a mechanical lever principle, the analog signal of capillary pressure Pm=k*F, where k represents the lever leverage, and F represents the pressure of the pressure sensor) to achieve the measurement of the capillary pressure (Pₘ), and the capillary pressure is converted into the form of voltage through the signal acquisition and amplification circuit 15 and then outputted. The outputted voltage signal is transferred to the data processing and analyzing module 4 through the signal acquisition and amplification circuit 15, thereby achieving display of numerical values and waveforms. The precision precession device 11 includes a precision screw and a precision nut.

As shown in FIG. 2, the microcirculation microscopic image capturing device 3 includes a microscope 31 and a camera 32, one end of the microscope 31 directly faces the capillary-pressure measuring device 13 of the capillary-pressure detection device 1, the other end of the microscope 31 directly faces the camera, and the camera 32 is connected to the data processing and analyzing module 4; the microscope 31 is used for amplifying the capillary to obtain an enlarged capillary, and the camera 32 is used for acquiring a static circulation image and a dynamic circulation image of the enlarged capillary, and output the static circulation image and the dynamic circulation image to the data processing and analyzing module 4. The data processing and analyzing module 4 acquires a capillary blood flow velocity v and a vascular diameter D according to the static circulation image and the dynamic circulation image, depicts a vascular network, and further calculates the single-blood-vessel blood flow volume Q and a local blood flow volume Qᵤ. Particularly, the measurement of the capillary blood flow velocity v is conducted by employing a dynamic-image playback technology, in a manner of fast-acquisition-slow-playback (high-speed camera), frame by frame identification, and man-machine conversation. That is, the blood flow velocity is measured by applying an automatic stepping method. The specific measurement method is as follows: assuming that a marker appears in the blood vessel of a certain frame of image (e.g., red blood cell aggregation-grain flow or single white blood cell-linear flow), the position coordinates (X₀, Y₀) of the marker are established via a computer mouse, then the image is played back frame by frame to observe the moving track of the marker to the position coordinates (X₁, Y₁) in the Nth frame of image, and therefore the blood flow velocity v=L/(N·t₀) (t₀ is the acquisition time of each frame of image) is obtained by calculating a distance L between two points and the number N of play-backed frames of images. This method not only can measure the grain flow velocity, but also can measure the linear flow velocity, and the measured value is an absolute blood flow velocity (m/s).

The measurement of the vascular diameter D is detection of capillary distribution of the nailfold microcirculation and detection of blood vessel edges through computer image processing and measurement technologies. The computer automatically measures the diameter D of the blood vessel and obtains a vascular network index by delineating a region of interest; for example parameters such as a vascular density Dₙ (vascular density—the number of blood vessels per unit length), a dynamic to static ratio K (the ratio of the number of flowing blood vessels to the total number of blood vessels in the region of interest), a replacement index P (the number of flowing blood vessels reopened after blocking of blood vessels to the number of flowing blood vessels before blocking of blood vessels), etc.

The single-blood-vessel blood flow volume Q (Q=v(πD²/4)) and the local blood flow volume Qᵤ=Q·Dₙ can be obtained from the capillary blood flow velocity v measured above, and the capillary resistance index R is obtained by calculating an equation (R=Q/Pₘ).

The segmental blood pressure measuring device 2 includes a controller (the controller is a single chip), an electronic valve, and multiple cuffs; the controller is connected to the control end of the electronic valve, the output end of the electronic valve is connected to the multiple cuffs respectively, and the controller is used for controlling the on-off actuation state of the electronic valve, and in turn controlling the multiple cuffs to conduct sequential measurement of a finger-artery blood pressure, a wrist-artery blood pressure and a brachial-artery blood pressure; the multiple cuffs are respectively connected to the controller, the multiple cuffs are used for measuring the finger-artery blood pressure, the wrist-artery blood pressure and the brachial-artery blood pressure, and outputting the finger-artery blood pressure, the wrist-artery blood pressure and the brachial-artery blood pressure to the controller; the controller is connected to the data processing and analyzing module 4, the controller is used for outputting the finger-artery blood pressure, the wrist-artery blood pressure and the brachial-artery blood pressure to the data processing and analyzing module 4. The data processing and analyzing module 4 evaluates the elasticity and resistance conditions of respective segmental blood vessels based on the finger-artery blood pressure, the wrist-artery blood pressure and the brachial-artery blood pressure. Particularly, in order to reduce a system error, the segmental blood pressure measuring device of the present invention employs a single blood pressure measuring module to perform blood-pressure measurement, where the blood pressure measuring module is connected to multiple cuffs through an electronic valve, the on-off actuation state of the electronic valve is controlled by the controller, and the controller is a single chip. By using a single chip automatic control technology, the blood-pressure measurement sequence of respective segments (finger-artery blood pressure, wrist-artery blood pressure, and brachial-artery blood pressure) and the measurement interval times (15 minutes) are controlled to automatically measure the brachial-artery blood pressure, the wrist-artery blood pressure and the finger-artery blood pressure in a time-sharing manner, and to upload the measurements to the data processing and analyzing module 4. The data processing and analyzing module 4 calculates the pressure differences of respective segments respectively, so as to obtain the pressure differences between arteries to respective segmental blood vessels of the capillary, which include the pressure difference between the brachial artery to the wrist artery, the pressure difference between the wrist artery to the finger artery, and the pressure difference between the finger artery to the capillary, for evaluating the elasticity and resistance conditions of respective segmental blood vessels.

The analysis of microvascular hemodynamic parameters in the present invention achieves acquisition and analysis of the capillary-pressure signal, the segmental-blood signal static circulation image and the dynamic circulation image. Indexes such as the capillary pressure Pm, the capillary blood flow velocity v, the capillary resistance R, the finger-artery blood pressure, the wrist-artery blood pressure, the brachial-artery blood pressure, and the blood pressure differences between respective segments are acquired through data processing, and the processing results are shown in the form of a graph. Finally, the results of the acquisition and analysis are printed in the form of a report through a printer, and diagnosis results and recommendations are given.

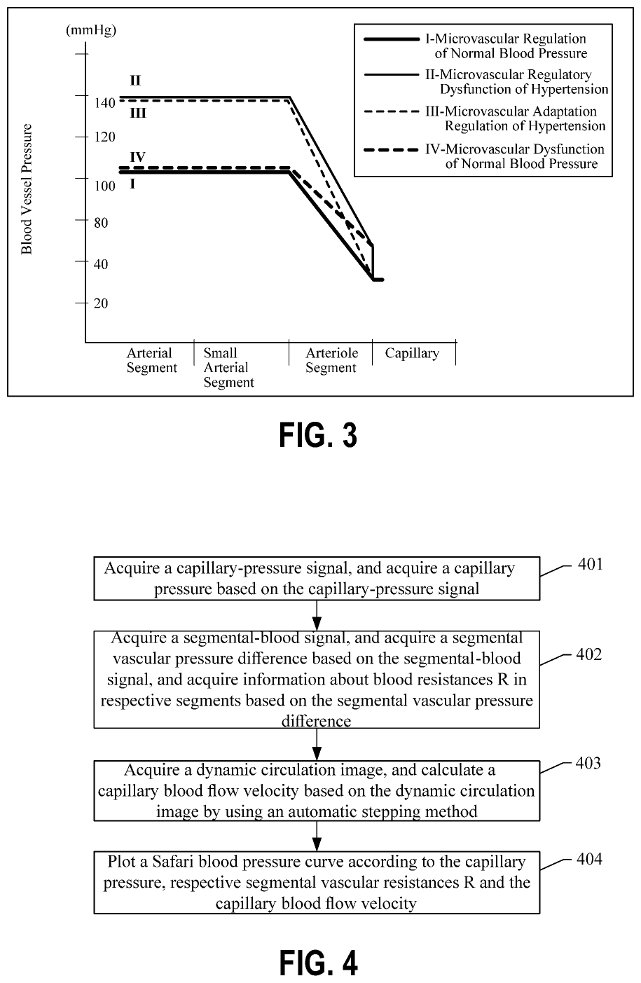

Main technical indexes for analysis of microvascular hemodynamic parameters in the present invention include:
(I) System indexes
(1) The measurement precision of pressure is 0.5 mmHg, the measurement precision of vascular radius is 0.01 um, the image acquisition speed is 200 frames/second, and the image resolution is 1280×1024.
(II) Hemodynamic indexes (1) the capillary pressure, the brachial-artery blood pressure, the wrist-artery blood pressure, and the finger-artery blood pressure are determined, an improved Safari blood pressure curve is depicted indirectly as shown in FIG. 3, with the vertical coordinate being the value of blood-vessel pressure, where I is a microvascular regulation curve of a normal blood pressure, II is a microvascular regulatory dysfunction curve of hypertension, III is a microvascular adaptation regulation curve of hypertension, and IV is a microvascular regulatory dysfunction curve of the normal blood pressure. The type of hypertension is determined by the curve, so as to select a targeted treatment plan.
(2) By using the single chip automatic control technology, the brachial-artery blood pressure, the wrist-artery blood pressure, and the finger-artery blood pressure are measured automatically, and uploaded to an upper computer, then the pressure differences of respective segments are calculated respectively, so as to obtain the pressure differences between arteries to respective segmental blood vessels of the capillary, which include the pressure difference between the brachial artery to the wrist artery, the pressure difference between the wrist artery to the finger artery, and the pressure difference between the finger artery to the capillary. The segmental pressure drop in a peripheral artery (an artery, a small artery and a precapillary arteriole, and in this system the segmental pressure drop is the pressure difference between the brachial artery, the wrist artery, and the finger-artery blood pressure—segmental pressure difference) is observed, and the absolute value of the total peripheral arterial pressure drop (resistance) (the total pressure difference=the brachial-artery blood pressure minus the capillary pressure) and the maximum pressure-drop distribution percentage (the maximum ratio among the ratios of respective segmental pressure differences to a total pressure difference) are calculated. Sites where the angiosclerosis or resistance will occur are explored to intervene in advance.
(3) Acquisition of microcirculation microscopic images and acquisition of hemodynamic indexes for pressure, flow and resistance. Microcirculation hemodynamic indexes include the capillary pressure, the blood flow velocity, the vascular network (density), and the blood flow resistance. The system obtains clear static and dynamic images of microcirculation through a microcirculation micrography system, by using a high-precision fast camera shooting technology. The measurement of the blood flow indexes is conducted by employing a dynamic-image playback technology, in a manner of fast-acquisition-slow-playback (high-speed camera), frame by frame identification, and man-machine conversation. That is, the blood flow velocity is measured by applying an automatic stepping method. The specific measurement method is as follows: assuming that a marker appears in the blood vessel of a certain frame of image (e.g., red blood cell aggregation-grain flow or single white blood cell-linear flow), the position coordinates (X₀, Y₀) of the marker are established via a computer mouse, then the image is played back frame by frame through the computer to automatically detect the moving track of the marker to the position coordinates (X₁, Y₁) in the next frame of image, and therefore the blood flow velocity v=L/(N·t₀) (t₀ is the acquisition time of each frame of image) is obtained by calculating a distance L between two points and the number N of play-backed frames of images. This method not only can measure the grain flow velocity, but also can measure the linear flow velocity, and the measured value is an absolute blood flow velocity (m/s). The measurement of a microcirculation static index is detection of capillary distribution of the nailfold microcirculation and detection of blood vessel edges through computer image processing and measurement technologies. The computer automatically measures the diameter D of the blood vessel and obtains a vascular network index by delineating a region of interest. Parameters such as a vascular density Dn (vascular density—the number of blood vessels per unit length), a dynamic to static ratio K (the ratio of the number of flowing blood vessels to the total number of blood vessels in the region of interest), a replacement index P (the number of flowing blood vessels reopened after blocking of blood vessels to the number of flowing blood vessels before blocking of blood vessels), etc. The single-blood-vessel blood flow volume Q (Q=v(πD²/4)) and the local blood flow volume Qᵤ (Qᵤ=Q·Dₙ) can be obtained from the blood flow velocity measured above, and the capillary resistance index R is obtained by calculating an equation (R=Q/Pₘ).
(4) Measurement of microcirculation blood flow reserve and endothelial functions.

A manner is employed, in which the finger artery is blocked (bound by a rubber band) to observe the blood flow stopping of the nailfold microcirculation, and 5 minutes later the blocking of the finger artery is removed to observe the blood flow changing conditions of the nailfold microcirculation. The nailfold capillary density (the number of blood vessels per unit length) is employed to evaluate the reactive hyperemia after arterial occlusion and calculate a capillary replacement percentage (the ratio of the number of flowing blood vessels reopened after blocking of blood vessels to the number of flowing blood vessels before blocking of blood vessels), reflecting and the blood flow reserve function and endothelial function. Mean volumetric blood flow of the nailfold capillary is used as a microcirculation perfusion blood flow index for observing the reactive hyperemia after arterial occlusion and evaluating the blood flow reserve function.

### Embodiment 3

In a further embodiment, a method is provided for analyzing microvascular hemodynamic parameters. As shown in FIG. 4, the analysis method includes the following steps: step 401, acquiring a capillary-pressure signal, and acquiring a capillary pressure Pₘ based on the capillary-pressure signal; step 402, acquiring a segmental-blood signal, and acquiring a segmental vascular pressure difference based on the segmental-blood signal, and acquiring information about blood resistances R in respective segments based on the segmental vascular pressure difference; step 403, acquiring a dynamic circulation image, and calculating a capillary blood flow velocity v based on the dynamic circulation image by using an automatic stepping method; and step 404, plotting a Safari blood pressure curve according to the capillary pressure Pₘ, respective segmental vascular resistances R and the capillary blood flow velocity v.

An improved Safari blood pressure curve is depicted indirectly as shown in FIG. 3, with the vertical coordinate being the value of blood-vessel pressure, where I is a microvascular regulation curve of a normal blood pressure, II is a microvascular regulatory dysfunction curve of hypertension, III is a microvascular adaptation regulation curve of hypertension, and IV is a microvascular regulatory dysfunction curve of the normal blood pressure. The type of hypertension is determined by the curve, so as to select a targeted treatment plan.

### Embodiment 4

In yet another embodiment, a method is provided for analyzing microvascular hemodynamic parameters. In the method, and with reference to FIG. 4, acquiring a segmental vascular pressure difference based on the segmental-blood signal, and acquiring information about blood resistances R in respective segments based on the segmental vascular pressure difference as described in step 402 specifically includes: calculating the segmental vascular pressure difference between arteries to the capillary based on the segmental-blood signal, where the segmental vascular pressure difference includes the pressure difference between the brachial artery and the wrist artery, the pressure difference between the wrist artery and the finger artery, and the pressure difference between the finger artery and the capillary; and evaluating the segmental vascular elasticity and the segmental vascular resistance based on the segmental vascular pressure difference. Particularly, the segmental pressure drop in a peripheral artery (an artery, a small artery and a precapillary arteriole, and in this system the segmental pressure drop is the pressure difference between the brachial artery, the wrist artery, and the finger-artery blood pressure—segmental pressure difference) is observed, and the absolute value of the total peripheral arterial pressure drop (resistance) (the total pressure difference=the brachial-artery blood pressure minus the capillary pressure) and the maximum pressure-drop distribution percentage (the maximum ratio among the ratios of respective segmental pressure differences to a total pressure difference) are calculated. Sites where the angiosclerosis or resistance will occur are explored to intervene in advance.

Calculating the capillary blood flow velocity v based on the dynamic circulation image by using an automatic stepping method described in step 403 specifically includes: acquiring a marker appearing in the dynamic circulation image; determining the original coordinate of the marker; delineating the motion track of the marker; playing back the dynamic circulation image frame by frame, and tracking the coordinate of the marker when the image is played back to the Nth frame based on the motion track, so as to obtain the coordinate of the marker in the Nth frame; calculating a distance L between the original coordinate and the coordinate of the marker in the Nth frame; and calculating a capillary blood flow velocity v utilizing a equation v=L/(N·t₀) based on the distance, where t₀ is the image acquisition time per frame. The capillary blood flow velocity reflects a single index of capillary hemodynamics. The extent of the capillary blood flow velocity is not enough to illustrate the microcirculation structure and the function state, and the capillary blood flow velocity should be combined with three indexes, i.e., the vascular network, the vascular pressure and the vascular resistance, to conduct comprehensive analysis.

After acquiring the dynamic circulation image, and calculating the capillary blood flow velocity v based on the dynamic circulation image by using an automatic stepping method described in step 403, it further includes: acquiring a static circulation image, and measuring a vascular diameter D and a vascular length based on the static circulation image, and delineating a vascular network; and obtaining a vascular density Dₙ, a dynamic to static ratio K, and a replacement index P based on the vascular network; calculating a single-blood-vessel blood flow volume Q utilizing an equation Q=v(πD²/4) based on the blood flow velocity v and the vascular diameter D; and calculating a local blood flow volume Qᵤ utilizing an equation Qᵤ=Q·Dₙ based on the single-blood-vessel blood flow volume Q and the vascular density Dₙ.

After step 404, it further includes acquiring indexes such as the capillary pressure Pm, the capillary blood flow velocity v, the capillary resistance R, the finger-artery blood pressure, the wrist-artery blood pressure, the brachial-artery blood pressure, and the blood pressure differences between respective segments through data processing, and showing the processing results in the form of a graph. Finally, the results of the acquisition and analysis are printed in the form of a report through a printer, and diagnosis results and recommendations are given.

Indexes such as the capillary blood flow velocity, the blood pressure and the resistance, each reflects a single index of capillary hemodynamics. Such a single index is not enough to illustrate the microcirculation structure and the function state, and it should be combined with multiple indexes such as the vascular network, the blood flow velocity, the blood flow volume, the blood pressure and the resistance, to conduct comprehensive analysis. If the blood flow velocity is increased, the blood pressure is increased, and the resistance is increased, then a high blood flow which damages the blood vessel is caused. However, high blood flow and low blood pressure are conducive to microcirculation perfusion. For the vascular network, high vascular density and parallel blood vessels facilitate blood perfusion.

Several examples are used for illustration of the principles and implementation methods of the present invention. The description of the embodiments is used to help illustrate the method and its core principles of the present invention. In addition, those skilled in the art can make various modifications in terms of specific embodiments and scope of application in accordance with the teachings of the present invention. In conclusion, the content of this specification shall not be construed as a limitation to the invention.

The embodiments described above are only descriptions of preferred embodiments of the present invention, and do not intended to limit the scope of the present invention. Various variations and modifications can be made to the technical solution of the present invention by those of ordinary skills in the art, without departing from the design and spirit of the present invention. The variations and modifications should all fall within the claimed scope defined by the claims of the present invention.

What is claimed is:

1. An analyzer for analyzing microvascular hemodynamic parameters, comprising:
a capillary-pressure detection device,
a segmental blood pressure measuring device,
a microcirculation microscopic image capturing device, and
a data processing and analyzing module;
wherein the capillary-pressure detection device is connected to the data processing and analyzing module to measure a capillary pressure, obtain a capillary-pressure signal, and output the capillary-pressure signal to the data processing and analyzing module;
the segmental blood pressure measuring device is connected to the data processing and analyzing module to acquire a finger-artery blood pressure, a wrist-artery blood pressure and a brachial-artery blood pressure, obtain a segmental-blood signal, and output the segmental-blood signal to the data processing and analyzing module;
the microcirculation microscopic image capturing device is connected to the data processing and analyzing module to acquire a static circulation image and a dynamic circulation image, and output the static circulation image and the dynamic circulation image to the data processing and analyzing module; and
the data processing and analyzing module is used to acquire hemodynamic parameters from the capillary-pressure signal, the segmental-blood signal, the static circulation image and the dynamic circulation image, and plot a Safari blood pressure curve; and the hemodynamic parameters comprise a capillary pressure Pₘ, a capillary blood flow velocity v, a capillary blood flow volume, a capillary local blood flow volume, a capillary resistance R, a segmental vascular pressure, and a segmental vascular pressure difference.

2. The analyzer of claim 1, wherein the capillary-pressure detection device comprises:
a precision precession device,
a precision lever,
a capillary-pressure measuring device,
a precision pressure sensor, and
a signal acquisition and amplification circuit;
wherein the precision precession device is connected to one end of the precision lever, the precision pressure sensor is disposed at one end of the precision lever, the capillary-pressure measuring device is disposed at the other end of the precision lever, and the precision precession device is used for pulling up one end of the precision lever, such that the other end of the precision lever squeezes the capillary on the capillary-pressure measuring device;
an output end of the precision pressure sensor is connected to an input end of the signal acquisition and amplification circuit, and the precision pressure sensor measures the capillary pressure of the capillary according to a mechanical lever principle, obtains a capillary-pressure analog signal, and output the capillary pressure analog signal to the signal acquisition and amplification circuit; and
an output end of the signal acquisition and amplification circuit is connected to the data processing and analyzing module, and the signal acquisition and amplification circuit is used to conduct amplifying and digital processing of the capillary pressure analog signal, obtain a capillary-pressure signal, and output the capillary-pressure signal to the data processing and analyzing module.

3. The analyzer of claim 2, wherein the precision precession device comprises a precision screw and a precision nut.

4. The analyzer of claim 2, wherein the microcirculation microscopic image capturing device comprises a microscope and a camera, wherein one end of the microscope directly faces the capillary-pressure measuring device of the capillary-pressure detection device, the camera is connected to the data processing and analyzing module, another end of the microscope directly faces the camera; the microscope is used for amplifying the capillary to obtain an enlarged capillary, the camera is used for acquiring a static circulation image and a dynamic circulation image of the enlarged capillary, and output the static circulation image and the dynamic circulation image to the data processing and analyzing module.

5. The analyzer of claim 1, wherein the segmental blood pressure measuring device comprises a controller, an electronic valve, and multiple cuffs;
Wherein the controller is connected to a control end of the electronic valve, an output end of the electronic valve is connected to the multiple cuffs respectively, and the controller is used for controlling an on-off actuation state of the electronic valve, and in turn controlling the multiple cuffs to conduct sequential measurement of a finger-artery blood pressure, a wrist-artery blood pressure and a brachial-artery blood pressure;
the multiple cuffs are respectively connected to the controller, the multiple cuffs are used for measuring the finger-artery blood pressure, the wrist-artery blood pressure and the brachial-artery blood pressure, and outputting the finger-artery blood pressure, the wrist-artery blood pressure and the brachial-artery blood pressure to the controller; and
the controller is connected to the data processing and analyzing module, and the controller is used for outputting the finger-artery blood pressure, the wrist-artery blood pressure and the brachial-artery blood pressure to the data processing and analyzing module.

6. A method for analyzing microvascular hemodynamic parameters, comprising:
acquiring a capillary-pressure signal, and acquiring a capillary pressure Pₘ based on the capillary-pressure signal;
acquiring a segmental-blood signal, and acquiring a segmental vascular pressure difference based on the segmental-blood signal, and acquiring information about blood resistances R in respective segments based on the segmental vascular pressure difference;
acquiring a dynamic circulation image, and calculating a capillary blood flow velocity v based on the dynamic circulation image by using an automatic stepping method; and
plotting a Safari blood pressure curve according to the capillary pressure Pₘ, respective segmental vascular resistances R and the capillary blood flow velocity v.

7. The method of claim 6, wherein the step of calculating the capillary blood flow velocity v based on the dynamic circulation image by using an automatic stepping method particularly comprises:
acquiring a marker appeared in the dynamic circulation image;
determining original coordinates of the marker;
delineating a motion track of the marker;
playing back the dynamic circulation image frame by frame, and tracking the coordinate of the marker when the image is played back to an Nth frame based on the motion track, so as to obtain the coordinate of the marker in the Nth frame;
calculating a distance L between the original coordinate and the coordinate of the marker in the Nth frame;
and calculating a capillary blood flow velocity v utilizing an equation v=L/(N·t₀) based on the distance, wherein t₀ is an image acquisition time per frame.

8. The method of claim 6, wherein after acquiring the dynamic circulation image, and calculating the capillary blood flow velocity v based on the dynamic circulation image by using an automatic stepping method, the method further comprises:
acquiring a static circulation image, and measuring a vascular diameter D and a vascular length based on the static circulation image, and delineating a vascular network.

9. The method of claim 8, wherein after acquiring the static circulation image, and measuring the vascular diameter D and the vascular length based on the static circulation image, and delineating the vascular network, the method further comprises:
obtaining a vascular density Dₙ, a dynamic to static ratio K, and a replacement index P based on the vascular network.

10. The method of claim 9, wherein after obtaining the vascular density Dₙ, the dynamic to static ratio K, and the replacement index P based on the vascular network, the method further comprises:
calculating a single-blood-vessel blood flow volume Q utilizing an equation Q=v(πD²/4) based on the blood flow velocity v and the vascular diameter D; and
calculating a local blood flow volume Qᵤ utilizing an equation Qᵤ=Q·Dₙ based on the single-blood-vessel blood flow volume Q and the vascular density Dₙ.
